(19) United States
(12) Patent Application Publication
(10) Pub. No.: US 2025/0381065 A1
(43) Pub. Date: Dec. 18, 2025

(54) Moisture Chamber Adapted For Use With Eyeglasses

(71) Applicant: BRITER EYEZ LLC, Amherst, NY (US)

(72) Inventors: Bridgette Cassety, Amherst, NY (US); Terry S. Galanis, JR., Marilla, NY (US); Ryan Unger, Bowmansville, NY (US)

(73) Assignee: Briter Eyez LLC, Amherst, NY (US)

(21) Appl. No.: 19/183,068

(22) Filed: Apr. 18, 2025

Related U.S. Application Data
(60) Provisional application No. 63/658,899, filed on Jun. 12, 2024.

(51) Int. Cl. A61F 9/00; G02C 11/00

(52) U.S. Cl. CPC A61F 9/0008; G02C 11/00; A61F 2009/0043

(57) ABSTRACT

A moisture chamber adapted for use with eyeglasses. The moisture chamber is a single-use, disposable, component that is temporarily adhered to the inside of a the lens of a pair of glasses. The moisture chamber may be used on one or both of the lenses and works by creating a moist environment for the eye to help alleviate dry eyes. The moisture chamber may be made of silicon and may be 3-D printed.

### CROSS-REFERENCE TO RELATED APPLICATIONS

This application claims priority to U.S. Provisional Patent Application having Ser. No. 63/658,899, filed on Jun. 12, 2024, the entire disclosure of which is hereby incorporated herein by reference.

### BACKGROUND

The field of optometry and ophthalmology addresses numerous eye conditions, including dry eye syndrome, which is characterized by insufficient lubrication and moisture on the ocular surface. Dry eye syndrome can be exacerbated by environmental factors such as low humidity and wind exposure, as well as prolonged visual tasks that reduce blink frequency, including reading and computer use.

Traditional solutions for managing dry eye syndrome include artificial tear solutions, prescription eye drops, and surgical interventions like punctal plugs. However, these treatments often provide only temporary relief and may be inconvenient or invasive. The need for a flexible, non-intrusive solution becomes apparent, especially for individuals experiencing dry eye symptoms in specific environments or during particular activities.

Existing integrated moisture chambers, often designed as part of specialty eyeglasses, aim to alleviate dry eye symptoms by creating a sealed environmental barrier around the eyes. Despite their efficacy, these devices are often permanent and require users to switch their regular eyeglasses with those equipped with moisture chambers. This approach can be cumbersome, expensive, and inconvenient, as permanently affixed moisture chambers may not be needed or desired at all times.

Patents such as Humphreys (U.S. Pat. No. 5,203,557) and Blanchet (U.S. Pat. No. 5,765,077) attempt to address these issues with various designs involving removable elements and specific eyeglass frames. However, these solutions still require modifications to existing frames or the use of specialized eyeglass frames, limiting their flexibility and user adaptability.

Recent advancements in material science and 3D printing technologies have paved the way for more innovative approaches. Flexible and biocompatible silicone materials offer a balance of comfort and functionality suitable for prolonged skin contact. Concurrently, 3D printing enables the precise fabrication of custom-fit designs, enhancing the versatility and adaptability of new product offerings in this space.

What is needed is a portable, disposable, and versatile moisture chamber solution that can seamlessly integrate with existing eyeglass configurations. This solution must balance effectiveness, convenience, and affordability, allowing users to efficiently address dry eye symptoms as needed without the drawbacks of current permanent or cumbersome designs. It should leverage advancements in material science and manufacturing technologies to provide a user-friendly, adaptable, and cost-efficient alternative that meets the diverse needs of individuals suffering from dry eye syndrome.

### FIELD

The present technology pertains to the field of ophthalmic devices, specifically designed to address dry eye syndrome by utilizing a moisture chamber that can be attached to eyeglasses. The device emphasizes flexibility, portability, and disposability, employing materials and manufacturing methods that ensure adaptability to various eyeglass frames and lenses.

### SUMMARY

In one aspect, the technology pertains to a moisture chamber designed for attachment to eyeglasses. The moisture chamber is configured to create a controlled, moisture-enhanced environment around the eye, assisting individuals suffering from dry eye syndrome. The design emphasizes a flexible, portable, and disposable solution, leveraging a specially designed silicone component.

One objective of the technology is to provide a removable and versatile moisture chamber that can be easily adhered to and detached from various types of eyeglass lenses. This aspect ensures that users do not need to permanently modify their eyeglasses, allowing for greater flexibility in usage.

In an embodiment, the moisture chamber is constructed as a three-dimensional piece of silicone shaped as a half-circle with upward curving sides forming a convex apex. This configuration is intended to provide substantial coverage around the eye without obstructing vision. The base of the moisture chamber includes an adhesive layer protected by a peelable cover, which allows for easy attachment and removal.

Yet another object of the technology is to offer a disposable and hygienic solution. After use, the moisture chamber can be easily removed and discarded, minimizing the need for cleaning and maintenance. This disposable nature enhances the practicality and convenience for users, especially those who require frequent replacements.

In another embodiment, the silicone component of the moisture chamber can be produced either through 3D printing or traditional molding methods. The 3D printing approach provides precise control over the geometry and ensures reproducibility, while the molding process may be more efficient for large-scale production.

In yet another aspect, the moisture chamber is compatible with a wide range of eyeglass lens materials such as polycarbonate, high-index polymers, and glass. The adhesive has been tested to ensure it provides secure attachment across various lens materials without leaving residue upon removal.

One object of the device is to enhance user control and comfort by allowing selective application of the moisture chamber to one or both lenses as needed. This functionality is particularly useful for individuals who may only require moisture retention for one eye.

Furthermore, the silicone material used is hypoallergenic and non-irritating, making it suitable for prolonged contact with the skin. This aspect ensures safety and comfort for users, particularly for those with sensitive skin.

In yet another embodiment, the moisture chamber's optimization involved developing a shape that balances coverage and flexibility, ensuring it adheres well to diverse eyeglass geometries. The materials and adhesive were chosen after extensive testing to provide strong adhesion while allowing for easy removal without residue.

The versatility and universal compatibility of the moisture chamber underscore its potential applications in various environments where eye hydration is essential. This includes dry climates, workplaces with high air conditioning usage, and for individuals requiring occasional relief from dry eye symptoms.

The invention aims to offer a significant improvement in the field of ophthalmic devices, providing a user-friendly alternative to permanently attached moisture chambers. The features described ensure that the moisture chamber adapts to various eyeglass types and materials, making it a practical and broadly applicable solution for managing dry eye syndrome and dry eye symptoms.

### BRIEF DESCRIPTION OF THE DRAWINGS

FIG. 1 is a perspective view of a moisture chamber designed for use in combination with eyeglasses, illustrating various components including tabs, hinges, and adhesive layers.

FIG. 2 is a bottom view of a moisture chamber for use in combination with eyeglasses, illustrating various adhesive layers and hinge structures.

FIG. 3 is a front view of a moisture chamber illustrating the upper main body, lower main body, and center hinge.

FIG. 4 is an interior view drawing illustrating a moisture chamber featuring an upper main body, a center hinge, and a lower main body, along with their respective adhesive layers.

FIG. 5 is a detailed view of a connection between a lower tab and a lower hinge within a moisture chamber assembly.

FIG. 6 is a perspective view illustrating the components of a moisture chamber in combination with eyeglasses.

### DETAILED DESCRIPTION

The term “moisture chamber” refers to a device configured to create a controlled environment that enhances and maintains moisture around a specific area, often in medical or consumer applications. When related to ophthalmic devices, a moisture chamber is designed to be affixed to a surface, such as an eyeglass lens or frames, to form a semi-enclosed space adjacent to the eye. This configuration helps to increase local humidity, thereby alleviating conditions such as dry eye syndrome. The moisture chamber typically comprises components such as main bodies and adhesive layers, which enable secure attachment to various substrates, such as eyeglass lenses, while being easily removable. The device may leverage flexible materials, such as silicone, to conform closely to the contours of the attached surface and maintain a moisture-rich environment effectively.

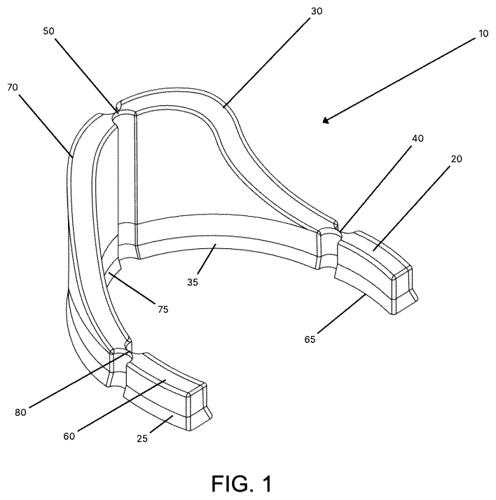

FIG. 1 shows a moisture chamber 10 designed for use with eyeglasses. Central to the structure is the upper main body 30, which is affixed by the upper hinge 40 to the upper tab 20. The upper tab 20 interfaces with an adhesive layer, designated as upper tab adhesive layer 65, to secure the upper portion to a surface, possibly the frame of the eyeglasses.

The upper main body 30 includes the upper main body adhesive layer 35, which facilitates attachment to additional components or surfaces. Extending from the upper section, the center hinge 50 connects the upper main body 30 to the lower main body 70, allowing for articulated movement between these sections. This articulation ensures flexible adjustments of the chamber relative to the eyeglasses.

The lower section of the chamber comprises the lower main body 70, which is further connected by the lower hinge 80 to the lower tab 60. The lower tab 60 is equipped with a lower tab adhesive layer 25, enhancing the attachment capability to various surfaces. Both the lower main body 70 and the lower main body adhesive layer 75 work in tandem, allowing for secure placement of the structure while maintaining positional integrity relative to the eyeglasses.

The upper main body 30 of the moisture chamber 10 is generally configured as a smoothly contoured piece with an arcuate edge that aligns with the curvature of the eyeglass lens. It has a concave profile that follows the surface of the lens, allowing for close adherence facilitated by the upper main body adhesive layer 35. This shaping ensures minimal obstruction to the user's vision while providing adequate coverage to maintain moisture around the eye area.

The lower main body 70 is shaped similarly, forming a complementary structure to the upper main body 30, with a curvature that aligns with the lower portion of the eyeglass lens. This design is intended to follow the natural contours of the lens and cheek, allowing the lower main body adhesive layer 75 to affix securely while maintaining a snug fit that aids in moisture retention around the eye. The curvature of both the upper and lower main bodies contributes to a resultant half-circle form, with upwards curving sides that meet at a convex apex, enhancing the chamber's ability to create a controlled environment for moisture retention.

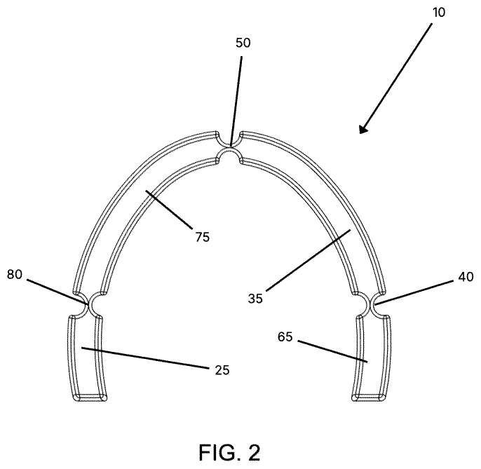

FIG. 2 shows a moisture chamber 10 designed for use in combination with eyeglasses. The moisture chamber 10 comprises several components that facilitate secure attachment and adherence to the eyeglasses. The moisture chamber 10 is structured with an upper main body adhesive layer 35 and a lower main body adhesive layer 75, each designed to ensure effective and reliable adhesion.

The figure further illustrates an arrangement of hinges and adhesive layers. The upper hinge 40 and the lower hinge 80 are depicted to allow flexibility and movement while maintaining the integrity of the moisture chamber 10. At the center, a center hinge 50 is provided, which functions to offer additional flexibility and structural guidance during use or application.

Also illustrated are the upper tab adhesive layer 65 and the lower tab adhesive layer 25. These adhesive layers are positioned to provide supplemental adhesion points, ensuring stability when the moisture chamber 10 is applied to the eyeglasses. The various adhesive layers and hinge components may be varied in alternate embodiments to accommodate different eyeglass configurations or materials, enhancing the versatility of the moisture chamber 10.

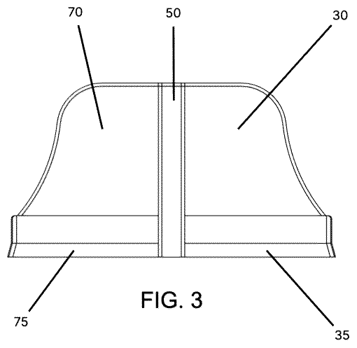

FIG. 3 shows a sectional view of a moisture chamber apparatus, designed for use in combination with eyeglasses. The representation illustrates a structure typically comprising an upper main body 30 and a lower main body 70, each corresponding to a portion of the moisture chamber that interfaces with the eyeglasses.

The upper main body 30 is depicted with an accompanying upper main body adhesive layer 35 along its lower edge, which facilitates attachment to the eyeglasses. This adhesive layer 35 may vary in composition or thickness to accommodate different types of eyeglass materials or designs. Additionally, the lower main body 70 features a lower main body adhesive layer 75 along its bottom portion, similarly enabling secure adherence to another section of the eyeglasses or to the facial structure of a user, depending on design variations.

A center hinge 50 is centrally located between the upper main body 30 and the lower main body 70, and has a pivotal connection that allows for the articulation or adjustment of the moisture chamber relative to the eyeglasses. The design of the center hinge 50 may be varied to offer different ranges of motion, durability under various usage conditions, and compatibility with different eyeglass frame designs.

In alternative embodiments, variations in the material or shape of the upper main body 30 and lower main body 70 may be introduced to enhance comfort, fit, or moisture retention capabilities, thereby offering adaptability across diverse consumer needs or preferences. The adhesive layers 35 and 75 may also be modified or substituted with non-adhesive fastening mechanisms, such as clips or straps, in alternate configurations.

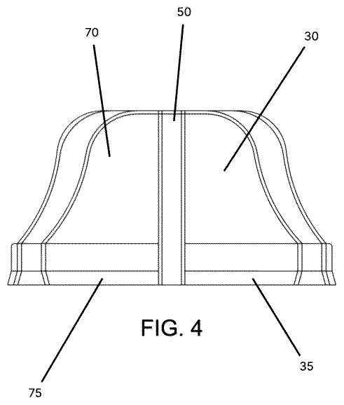

FIG. 4 shows a side elevation view of a moisture chamber designed for use in combination with eyeglasses. The configuration depicted includes an upper main body 30 and a lower main body 70, which are joined by a center hinge 50 that facilitates adjustable movement between the two portions. This hinge arrangement permits the moisture chamber to adapt to various eyeglass dimensions or shapes, enhancing compatibility and comfort.

The upper main body 30 is associated with an upper main body adhesive layer 35. This adhesive layer 35 allows for secure attachment to the upper section of eyeglasses, ensuring stability during use. Similarly, the lower main body 70 incorporates a lower main body adhesive layer 75, which provides an attachment mechanism to the lower portion of the eyeglasses. These adhesive layers can vary in material composition or adhesive properties to accommodate different applications or user preferences.

The center hinge 50 is strategically positioned to allow pivotal motion, which enables the moisture chamber to be folded or adjusted if necessary. Such adjustability could be beneficial for storage or transport and may facilitate a customized fit for the user. The integration of these components indicates a focus on maintaining moisture around the eyes, potentially improving comfort for eyeglass wearers in dry environments. Alternate embodiments might include variations in the hinge design or adhesive properties to cater to specific user needs or eyeglass designs.

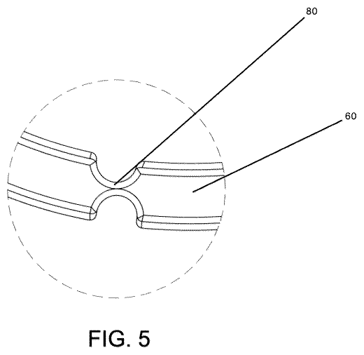

FIG. 5 shows a detailed view of a component of a moisture chamber for use in combination with eyeglasses. The figure specifically illustrates the interaction between a lower tab 60 and a lower hinge 80. The depicted configuration provides an interface where the lower tab 60 is structured to fit into or connect with the lower hinge 80, facilitating a pivotal or rotatable connection.

The lower tab 60 is designed in such a manner that it allows for secure engagement with the lower hinge 80, which may enable the moisture chamber to attach or detach from a corresponding component on the eyeglasses. The geometry and dimensions of lower tab 60 and lower hinge 80 ensure adequate functionality while maintaining the overall structural integrity necessary for repeated use.

Alternate embodiments of the moisture chamber may vary the shape, material, or method of connection between the lower tab 60 and lower hinge 80 to accommodate different eyeglass configurations. Such variations may include changes to the curvature or size of the lower tab 60 and adaptations in the design of the lower hinge 80 to optimize for different applications or enhance user convenience.

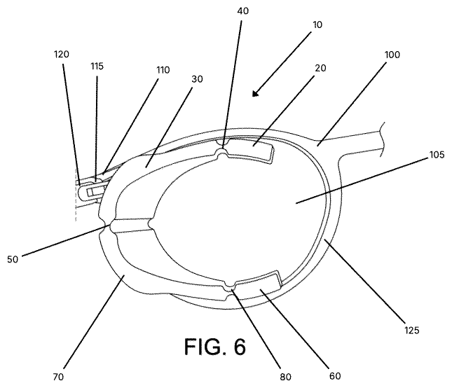

FIG. 6 shows a moisture chamber 10 designed for integration with eyeglasses 100. The moisture chamber 10 is structured to support the formation of a semi-enclosed environment around the lens 105, thereby enhancing comfort for the wearer. The chamber consists of an upper main body 30 and a lower main body 70, which are connected via a center hinge 50. The chamber 10 is affixed to the eyeglasses 100 and extends across the lens 105, with a focus on maintaining close contact.

The upper main body 30 is equipped with an upper tab 20, which interfaces with an upper hinge 40. This configuration facilitates selective pivoting or separation of the upper main body 30 from the lens 105 to allow for maintenance or adjustment. Complementarily, the lower main body 70 features a lower tab 60 connected to a lower hinge 80, performing a similar function as part of the overall structure. Also depicted is frame 125.

47. Additionally, the eyeglasses 100 incorporate an end piece 110 connected to an arm 120 through a hinge 115. This linkage allows for typical opening and closing movements of the arm 120, thereby ensuring secure positioning on the wearer's ears. The entire assembly depicted in FIG. 6 underscores the integration of the moisture chamber 10 with standard eyeglass components, allowing for variations in chamber design, material selection, and attachment mechanisms to suit different eyeglass models.

The moisture chamber 10 is constructed from silicone material and has a three-dimensional configuration designed to create a controlled, moisture-enhanced environment around the eye area. The moisture chamber 10 exhibits a half-circle shape, with the silicone material on either side curving upward to form a convex apex before symmetrically curving downward, adhering closely to the rim of the eyeglasses.

The various adhesive layers make contact with the interior side of the lens 105. The adhesive layers are engineered to secure the moisture chamber 10 firmly to the lens 105 while allowing for easy removal without leaving residue. The adhesive backing is initially protected by a peelable cover (not shown), which the user removes before attachment. In an alternative embodiment, the adhesive layers may make contact with the frames of the eye glasses instead of the lens.

The design accommodates various eyeglass frame geometries, ensuring the moisture chamber 10 conforms to different shapes and sizes. The moisture chamber 10 extends towards the eyeglass arms 120, effectively establishing a sealed perimeter around the eye region to maintain moisture retention. The dimensions and flexibility of the silicone material afford significant coverage without obstructing vision.

It is noted that in the Figs. above, the moisture chamber 10 is depicted in an orientation for use with the left lens of a pair of eyeglasses. It is understood that the moisture chamber 10 can easily be flipped so that it can be used in combination with the right lens of a pair of eyeglasses. In this orientation, the upper and lower components are reversed.

The above Figs. detail the moisture chamber 10 having a particular curvature and dimensions. The chamber exhibits a half-circle shape with upward-curved silicone sides that reach a convex apex before symmetrically curving downward. Smooth edging enhances the device's ergonomic design, ensuring comfort during use.

In other embodiments, the moisture chamber may be made of silicon, silicon blends, or any other suitable material. The moisture chamber may be 3-D printed or produced by a mold or any other suitable process of manufacturing.

In alternate embodiments, the moisture chamber 10 can be produced via 3D printing or traditional molding techniques to meet different production needs. These methods ensure precise control over the chamber's geometry and consistency in quality. Additionally, the silicone material selected for the moisture chamber 10 is hypoallergenic, making it safe for prolonged contact with the skin.

The described configuration enables the moisture chamber 10 to be easily attached or removed as needed, providing convenience and flexibility for users suffering from dry eye syndrome. The disposable nature of the moisture chamber 10 ensures hygiene and reduces maintenance, offering an effective moisture-retaining solution adaptable to a wide range of eyeglass designs.

The moisture chamber 10 is structured to create a sealed environment around the perimeter of the eyeglass lens 105, thereby maintaining a moisture-enhanced space near the user's eye.

As discussed above, the moisture chamber 10 is designed with multiple hinge points providing flexibility and allowing the chamber to conform closely to the curvature of the eyeglasses and lens 105. This adaptable design ensures that the moisture chamber 10 adheres seamlessly to various eyeglass configurations.

It will be appreciated by those skilled in the art that while the moisture chamber for use in combination with eyeglasses has been described in detail herein, the invention is not necessarily so limited and other examples, embodiments, uses, modifications, and departures from the embodiments, examples, uses, and modifications may be made without departing from the process and all such embodiments are intended to be within the scope and spirit of the appended claims.

1. A moisture chamber for use with eyeglasses, comprising:
an upper main body having an upper main body adhesive layer configured to adhere the upper main body to a surface;
a lower main body having a lower main body adhesive layer configured to adhere the lower main body to a surface;
a center hinge operatively connecting the upper main body to the lower main body;
wherein the moisture chamber is configured to attach to the interior of a lens of a pair of eyeglasses, creating a moisture-enhanced environment around the eye.

2. The moisture chamber of claim 1, wherein the upper main body and the lower main body are made from a hypoallergenic silicone material.

3. The moisture chamber of claim 1, further comprising a lower tab connected to the lower main body via a lower hinge, the lower tab including a lower tab adhesive layer for attachment to a surface.

4. The moisture chamber of claim 1, wherein the upper main body further comprises an upper tab connected to the upper main body via an upper hinge, the upper tab including an upper tab adhesive layer for attachment to a surface.

5. The moisture chamber of claim 1, wherein the moisture chamber is shaped as a half-circle with upward curving sides forming a convex apex.

6. The moisture chamber of claim 1, wherein the upper main body and the lower main body are configured with a concave profile, allowing each main body to closely adhere to the curvature of the lens.

7. The moisture chamber of claim 5, wherein the upper main body and the lower main body are configured with a concave profile, allowing each main body to closely adhere to the curvature of the lens.

8. The moisture chamber of claim 4, wherein the upper main body and the lower main body are configured with a concave profile, allowing each main body to closely adhere to the curvature of the lens.

9. The moisture chamber of claim 8, wherein the moisture chamber is shaped as a half-circle with upward curving sides forming a convex apex.

10. The moisture chamber of claim 1, wherein the upper main body adhesive layer and the lower main body adhesive layer are configured to enable secure attachment and easy removal from lens 105 without leaving residue.

11. The moisture chamber of claim 1, further comprising a removable peelable cover protecting the adhesive layers prior to attachment.

12. The moisture chamber of claim 1, wherein the adhesive layers are configured to adhere to various eyeglass lens materials, including polycarbonate, high-index polymers, and glass.

13. The moisture chamber of claim 1, configured to be repositioned between a left lens and a right lens of a pair of eyeglasses without losing adhesive efficacy.

14. The moisture chamber of claim 1, wherein the center hinge is configured to allow articulated movement of the upper and lower main bodies relative to each other, facilitating adjustment to fit varying eyeglass geometries.

15. The moisture chamber of claim 1, wherein the dimensions of the moisture chamber are configured to provide comprehensive coverage around the eye to maintain moisture retention without obstructing vision.

16. The moisture chamber of claim 1, wherein the moisture chamber is disposable, removable, and designed for single-use application, enhancing hygiene and user convenience.

17. A moisture chamber for use with eyeglasses, comprising:
an upper main body having an upper main body adhesive layer configured to adhere the upper main body to a surface;
a lower main body having a lower main body adhesive layer configured to adhere the lower main body to a surface;
a center hinge operatively connecting the upper main body to the lower main body;
a lower tab connected to the lower main body via a lower hinge, the lower tab including a lower tab adhesive layer for attachment to a surface;
an upper tab connected to the upper main body via an upper hinge, the upper tab including an upper tab adhesive layer for attachment to a surface;
wherein the moisture chamber is configured to attach to the interior of a lens of a pair of eyeglasses, creating a moisture-enhanced environment around the eye.

18. The moisture chamber of claim 17, wherein the upper main body and the lower main body are configured with a concave profile, allowing each main body to closely adhere to the curvature of the lens 105.

19. The moisture chamber of claim 17, wherein the upper main body and the lower main body are configured with upward curving sides forming a convex apex, enabling the moisture chamber to create a moisture-enhanced environment around the eye with enhanced contouring to the eyeglass lens.

20. The moisture chamber of claim 18, wherein the upper main body and the lower main body are configured with upward curving sides forming a convex apex, enabling the moisture chamber to create a moisture-enhanced environment around the eye with enhanced contouring to the eyeglass lens.
